(19) United States
(12) Patent Application Publication
(10) Pub. No.: US 2005/0042714 A1
(43) Pub. Date: Feb. 24, 2005

(54) Glycoconjugates of sialic acid derivates, methods for their production and use thereof

(76) Inventors: Michael Pawlita, Eschelborn (DE); Cornelia Oetke, Lueneburg (DE); Oliver Keppler, Weinheim (DE); Reinhard Brossmer, Neckargemund (DE); Stephan Hinderlich, Berlin (DE); Werner Reutter, Berlin (DE)

Correspondence Address: KRIEGSMAN & KRIEGSMAN, 665 FRANKLIN STREET, FRAMINGHAM, MA, 01702, US

(21) Appl. No.: 10/487,023

(22) PCT Filed: Aug. 17, 2002
(86) PCT No.: PCT/EP02/09198

(30) Foreign Application Priority Data
Aug. 17, 2001 (EP) 01250297.7

(51) Int. Cl. A61K 38/16; C12P 21/06; C07K 9/00; C07K 14/47

(52) U.S. Cl. 435/68.1; 514/8; 530/395; 435/320.1; 435/325; 530/322; 536/23.1; 536/53

(57) ABSTRACT

The present invention relates to glycoconjugates containing a sialic acid derivate of general formula (I) and wherein the sialic acid derivate of general formula (I) is conjugated to a mono-, di- or oligosaccharide with up to 40 glycosidically linked, optionally branched sugar residues representing furanose and/or pyranose rings, which are linked N- or O-glycosidically to the polypeptide. The sialic derivatives of general formula (I) are useful for producing pharmaceutical compositions for immunosuppression, cell protection, stimulation of hematopoesis regulation of hormonal secretion and hormonal activation.

The present invention relates to glycoconjugates of sialic acid derivates, methods for their production and use thereof.

Sialic acids are the most frequently found terminal mono-saccharides on the surface of eukaryotic cells and they perform important functions in biological recognition phenomena, including cell-cell interactions, and binding of viruses, bacteria, and parasites to their cellular receptors. Over 30 different naturally occuring members of this family of 9-carbon aminosugars have been identified up to now. The most abundant sialic acid in mammalian cells are N-acetyl neuraminic acid (NeuAc) and N-glycolyl neuraminic acid (NeuGc), with the exception of humans which lack NeuGc due to an enzyme deficiency. This loss of NeuGc and increase in NeuAc in humans may have altered biological processes as some adhesion molecules recognize glycoconjugates containing NeuGc and NeuAc with different affinities.

Many oncofetal antigens in mammals including humans carry sialic acid side chain modifications like 9-O-acetylation and N-glycolyl modifications. For example the unusual gangliosides O-acetyl-GD3 and N-glycolyl-GM3 and the mammary serum antigen (MSA) were found in breast tumors and GM2 containing NeuGc in human colon cancers. In contrast O-acetylation of sialyl Lewis X antigen (sLex) decreases from normal colonic mucosa to primary carcinomas and their liver metastases.

Furthermore, sialic acid species can determine the host range of pathogens like Influenza A or enterotoxic Escherichia coli strains (ETEC). For Influenza the binding specificity of hemaglutinin differs between isolates from different hosts, but correlates with the types of sialic acids expressed on host cells. The K99-fimbriae of ETEC bind to NeuGc-GM3 as a cellular receptor, but not to NeuAc-GM3 and therefore K99-bearing E. coli strains are non-pathogenic for humans and animals that do not express NeuGc.

From a therapeutic perspective, a group of sialic acid analogues, which were rationally designed as high-affinity inhibitors of influenza virus neuramindase, have recently been introduced successfully as an anti-flu medication in humans.

There are also some more general functions of sialic acid. They contribute significantly to the negative charge of cell surfaces and glycoproteins, causing repulsion of cells and influencing the physicochemical properties of glycoproteins and they can mask recognition epitopes. Sialylation determines the serum half-life of glycoproteins by preventing the removal of glycoproteins via the asialoreceptor in the liver.

Despite the diagnostic and therapeutic potential of sialic acid analogues, structure-function analyses have largely been confined to in vitro binding studies or competitive inhibition assays. The effect of synthetic sialic acid analogues as part of sialoglycoconjugate receptors in living cells could not be studied due to very low incorporation efficiencies using exogenous transfer methods.

Recombinant glycoproteins are well known and widely used. For example, WO 00/29567 A1 describes the use of neuramic acids derivates for the production of recombinant glycoproteins.

The drawback of the glycoproteins of the state of the art is that the method for production is time consuming and expensive. Many purification steps must be performed in order to obtain a substance in pharmaceutically acceptable quality.

WO 94/24167 A1 shows biosynthetic incorporation of sialic acids from sialic acid precursors (mannosamins) added to the cell culture medium. But modifications are limited to C5 (of the resulting sialic acid).

It is an object of the present invention to provide glycoconjugates like glycoproteins in a simple way, without the disadvantages of the state of the art.

This problem is solved according to the invention by providing glycoconjugates containing a sialic acid derivate of general formula I 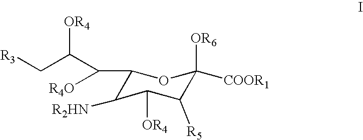
wherein
R1 represents hydrogen or lower alkyl up to 5 carbon atoms, which may be branched, unbranched, acyclic, alicyclic or cyclic,
R2 is acetyl, thioacetyl or succinyl, which can be substituted with up to 3 fluoro atoms or an amino group, or an acyl or thioacyl group with up to 5 carbon atoms in the alkyl moiety, which may be branched, unbranched, acyclic, alicyclic or cyclic,
R3 is a halogen atom, a methylsulfide group, a methylsulfate group or acyl or thioacyl group with up to 5 carbon atoms in the alkyl moiety, which may be branched, unbranched, acyclic, alicyclic or cyclic,
R4 represents, independently from each other, hydrogen, acetyl, thioacetyl or succinyl, which can be substituted with up to 3 fluoro atoms or an amino group, or an acyl or thioacyl group with up to 5 carbon atoms in the alkyl moiety, which may be branched, unbranched, acyclic, alicyclic or cyclic,
R5 is hydrogen or a halogen atom
R6 is a bond
   and wherein the sialic acid derivate of general formula I is conjugated via R6 to a mono-, di- or oligosaccharide with up to 40 glycosidically linked, optionally branched sugar residues representing furanose and/or pyranose rings,
   which are linked N- or O-glycosidically to the polypeptide.

According to the invention the glycoconjugates are obtainable by incorporating a sialic derivative of general formula I as given above, under the proviso that R6 represents hydrogen, acetyl, thioacetyl or succinyl, which can be substituted with up to 3 fluoro atoms or an amino group, or an acyl or thioacyl group with up to 5 carbon atoms in the alkyl moiety, which may be branched, unbranched, acyclic, alicyclic or cyclic,

to a living body, especially to a mammal and cells derived from mammals and lower eukaryotes.

Preferred are glycoconjugates

wherein

R1 represents hydrogen or lower alkyl up to 3 carbon atoms, which may be branched, unbranched or cyclic,
R2 is acetyl,
R3 represents, independently from each other, hydrogen or acetyl,
R4 represents, independently from each other, hydrogen or acetyl and
R5 is hydrogen.

Preferred are also glycoconjugates,

wherein

R1 represents hydrogen, methyl or ethyl,
R2 is acetyl,
R3 represents, independently from each other, hydrogen or acetyl,
R4 represents, independently from each other, hydrogen or acetyl and
R5 is hydrogen.

Especially preferred are glycoconjugates,

wherein the sialic acid derivate to be incorporated is selected from

5-N-acetyl-9-deoxy-neuraminic acid,
5-N-acetyl-9-deoxy-neuraminic acid methyl ester peracetylate,
5-N-acetyl-9-deoxy-neuraminic acid ethyl ester peracetylate,
5-N-acetyl-9-amino-9-deoxy-neuraminic acid,
5-N-acetyl-9-amino-9-deoxy-neuraminic acid methyl ester peracetylate,
5-N-acetyl-9-amino-9-deoxy-neuraminic acid ethyl ester peracetylate,
5-N-acetyl-9-acetamido-9-deoxy-neuraminic acid,
5-N-acetyl-9-acetamido-9-deoxy-neuraminic acid methyl ester peracetylate,
5-N-acetyl-9-acetamido-9-deoxy-neuraminic acid ethyl ester peracetylate,
5-N-acetyl-9-aminoacetamido-9-deoxy-neuraminic acid,
5-N-acetyl-9-aminoacetamido-9-deoxy-neuraminic acid methyl ester peracetylate,
5-N-acetyl-9-aminoacetamido-9-deoxy-neuraminic acid ethyl ester peracetylate,
5-N-acetyl-9-deoxy-9-succinylamido-neuraminic acid,
5-N-acetyl-9-deoxy-9-succinylamido-neuraminic acid methyl ester peracetylate,
5-N-acetyl-9-deoxy-9-succinylamido-neuraminic acid ethyl ester peracetylate,
5-N-acetyl-9-deoxy-9-iodo-neuraminic acid,
5-N-acetyl-9-deoxy-9-iodo-neuraminic acid methyl ester peracetylate,
5-N-acetyl-9-deoxy-9-iodo-neuraminic acid ethyl ester peracetylate,
5-N-acetyl-9-deoxy-9-thio-neuraminic acid,
5-N-acetyl-9-deoxy-9-thio-neuraminic acid methyl ester peracetylate,
5-N-acetyl-9-deoxy-9-thio-neuraminic acid ethyl ester peracetylate,
5-N-acetyl-9-deoxy-9-methylthio-neuraminic acid,
5-N-acetyl-9-deoxy-9-methylthio-neuraminic acid methyl ester peracetylate,
5-N-acetyl-9-deoxy-9-methylthio-neuraminic acid ethyl ester peracetylate,
5-N-acetyl-9-deoxy-9-methylsulfonyl-neuraminic acid,
5-N-acetyl-9-deoxy-9-methylsulfonyl-neuraminic acid methyl ester peracetylate,
5-N-acetyl-9-deoxy-9-methylsulfonyl-neuraminic acid ethyl ester peracetylate,
5-N-flouroacetyl-neuraminic acid methyl ester peracetylate,
5-N-flouroacetyl-neuraminic acid ethyl ester peracetylate,
5-N-triflouroacetyl-neuraminic acid methyl ester peracetylate,
5-N-triflouroacetyl-neuraminic acid ethyl ester peracetylate,
5-N-aminoacetyl-neuraminic acid methyl ester peracetylate,
5-N-aminoacetyl-neuraminic acid ethyl ester peracetylate,
5-N-succinyl-neuraminic acid methyl ester peracetylate,
5-N-succinyl-neuraminic acid ethyl ester peracetylate,
5-N-thioacetyl-neuraminic acid methyl ester peracetylate,
5-N-thioacetyl-neuraminic acid ethyl ester peracetylate,
5-N-acetyl-9-deoxy-9-iodo-neuraminic acid methyl ester,
5-N-acetyl-9-deoxy-9-iodo-neuraminic acid methyl ester peracetylate,
5-N-acetyl-9-deoxy-9-iodo-neuraminic acid ethyl ester,
5-N-acetyl-9-deoxy-9-iodo-neuraminic acid ethyl ester peracetylate.

According to the invention it is also possible and preferred to use the following residues for the groups R2, R4, R5 and R6 to form sialic derivates according to the present invention. Suitable residues are, but not limited to, deoxy, amino, acetamido, succinylamido, iodo, di-iodo, tri-iodo, fluoro, di-fluoro, tri-fluoro, chloro, di-chloro, tri-chloro, thio, methylthio, methylsulfonyl, benzoyl, phenyl, methyl, ethyl, propanoyl, butanoyl, pentanoyl, glycylamido, glycolyl, azido, fluoresceinisothiocyanat, benzamido, hexanoylamido, formyl, benzoyloxycarbonyl. It is further according to the invention that, if possible, two of the residues form a lacton and/or lactam structural group.

It is according to the present invention highly preferred that the hydroxy groups are acetylated. In the context of this application the term peracetylate does mean that all or nearly all residues R4 are acetyl groups.

It is further preferred that residue R1 is different from hydrogen. This means that on C1 an ester function is present. Most preferred are in the context of this invention methyl and ethyl esters.

Without any undue burden a person skilled in the art will find the suitable combination of residues by using the methods as described herein.

It is another object of the invention to provide a method for the production of glycoconjugates according to the invention, characterized in that a sialic derivative of general formula I 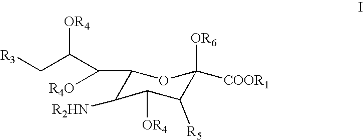
wherein
R1 represents hydrogen or lower alkyl up to 5 carbon atoms, which may be branched, unbranched, acyclic, alicyclic or cyclic,
R2 is acetyl, thioacetyl or succinyl, which can be substituted with up to 3 fluoro atoms or an amino group, or an acyl or thioacyl group with up to 5 carbon atoms in the alkyl moiety, which may be branched, unbranched, acyclic, alicyclic or cyclic,
R3 is a halogen atom, a methylsulfide group, a methylsulfate group or acyl or thioacyl group with up to 5 carbon atoms in the alkyl moiety, which may be branched, unbranched, acyclic, alicyclic or cyclic,
R4 represents, independently from each other, hydrogen, acetyl, thioacetyl or succinyl, which can be substituted with up to 3 fluoro atoms or an amino group, or an acyl or thioacyl group with up to 5 carbon atoms in the alkyl moiety, which may be branched, unbranched, acyclic, alicyclic or cyclic,
R5 is hydrogen or a halogen atom and
R6 represents hydrogen, acetyl, thioacetyl or succinyl, which can be substituted with up to 3 fluoro atoms or an amino group, or an acyl or thioacyl group with up to 5 carbon atoms in the alkyl moiety, which may be branched, unbranched, acyclic, alicyclic or cyclic,
   is incorporated into a living body, especially to a mammal or cells derived from mammals and lower eukaryotes.

Especially preferred are sialic acid derivates that are esterified and peracetylated as disclosed in the context of this invention.

It is another object of the invention to use a sialic derivative of general formula I as given above, for producing a pharmaceutical composition, for immunosuppression, cell protection, stimulation of hematopoesis regulation of hormonal secretion and hormonal activation.

Preferred are those sialic derivates wherein
R1 represents hydrogen or lower alkyl up to 5 carbon atoms, which may be branched, unbranched, acyclic, alicyclic or cyclic,
R2 is acetyl, thioacetyl or succinyl, which can be substituted with up to 3 fluoro atoms or an amino group, or an acyl or thioacyl group with up to 5 carbon atoms in the alkyl moiety, which may be branched, unbranched, acyclic, alicyclic or cyclic,
R3 is a halogen atom, a methylsulfide group, a methylsulfate group or acyl or thioacyl group with up to 5 carbon atoms in the alkyl moiety, which may be branched, unbranched, acyclic, alicyclic or cyclic,
R4 represents, independently from each other, hydrogen, acetyl, thioacetyl or succinyl, which can be substituted with up to 3 fluoro atoms or an amino group, or an acyl or thioacyl group with up to 5 carbon atoms in the alkyl moiety, which may be branched, unbranched, acyclic, alicyclic or cyclic,
R5 is hydrogen or a halogen atom and
R6 represents hydrogen, acetyl, thioacetyl or succinyl, which can be substituted with up to 3 fluoro atoms or an amino group, or an acyl or thioacyl group with up to 5 carbon atoms in the alkyl moiety, which may be branched, unbranched, acyclic, alicyclic or cyclic.

The pharmaceutical compositions according to the present invention are produced in a known manner using standard methods as known to those skilled in the art.

The present invention expands the repertoire for sialic acid variation in glycoconjugates.

It was surprisingly found that synthetic sialic acid analogues carrying C1, C3, C5, and C9 side chain modifications of different size, charge, and chemical properties can be taken up and incorporated into cellular glycoconjugates. This reveals a high degree of promiscuity of the sialic acid uptake and cellular metabolization pathway. The efficient incorporation of synthetic sialic analogues into living cells will facilitate submolecular analyses of sialic acid-dependent ligand-receptor interactions in their native context. More importantly, it opens a new way to generate secreted and non secreted, natural and recombinant glycoconjugates with modified sialic acids and thus altered biological and chemical characteristics and functions. So, it was surprising that the glycoconjugates of the present invention are easily formed in the cells.

In order to demonstrate this surprising finding the inventors used BJA-B K20 and HL60-I cells that are hyposialylated due to a UDP-GlcNAc 2-epimerase deficiency, a key enzyme of sialic acid biosynthesis. The fact that the hyposialylated cells have a defect in sialic acid biosynthesis makes them an ideal tool for the incorporation of modified sialic acid precursors, as analogues do not need to compete with endogenously synthesized sialic acids. It was found that medium supplementation with NeuAc complemented for endogenous hyposialylation in BJA-B K20 and HL60-I cells. NeuAc was rapidly taken up, metabolized, incorporated into cellular glycoconjugates, and exposed at the cell surface. This molecularly still uncharacterised uptake of NeuAc was active in all human cell lines and primary cells tested regardless of their prior sialylation status.

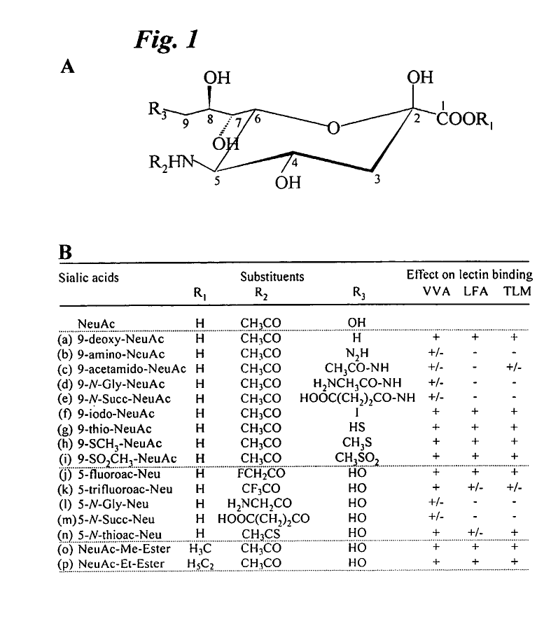

The present invention is explained in details using FIGS. 1 to 6.

FIG. 1

Synthetic sialic acid analogues used according to the invention. (A) All derivatives are based on NeuAc. (B) Sialic acids used are substituted either in position C1 (R1), C5 (R2) or C9 (R3). Summarized effects on lectin binding were arbitrarily set as strong (+), weak (+/−) and absent (−). Border values (+; +/−; −) were (≦0.75; ≦0.9; >0.9) for VVA, (≧4; ≧2; <2) for LFA and (≧2; ≧1.4; <1.4) for TLM.

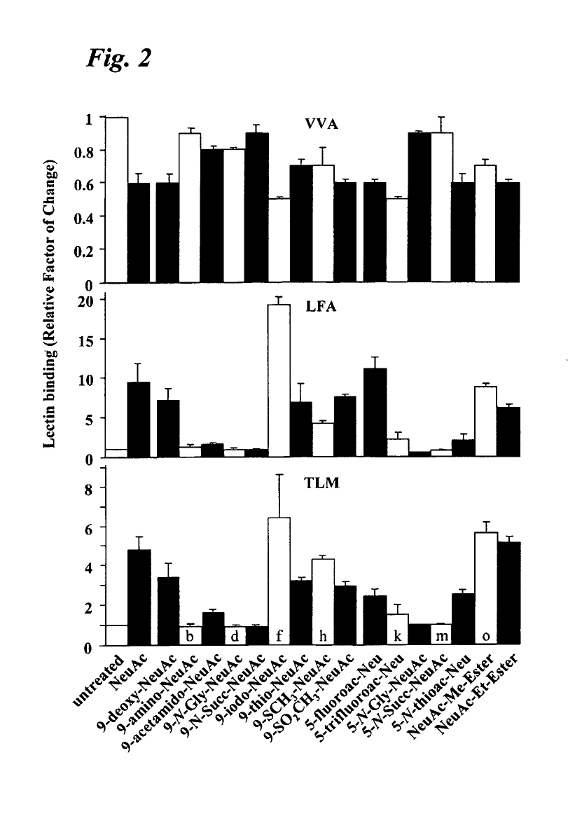

FIG. 2

Medium supplementation with some synthetic sialic acid analogues restores cell surface sialylation in hyposialylated HL60-I cells. Cells were cultivated in the presence of indicated synthetic sialic acid analogue (5 mM) for 24 h and stained with either directly fluorochrome-coupled lectins (VVA, LFA) or biotin-coupled lectin (TLM) and streptavidin-FITC and analyzed by flow cytometry. Values are given as “relative factor of change” (RFC) of the “mean fluorescence intensity” (MFI) of sialic acid-treated cells relative to untreated cells. The bars represent arithmetic means±SD (n³4) of one (x) or ≧two experiments.

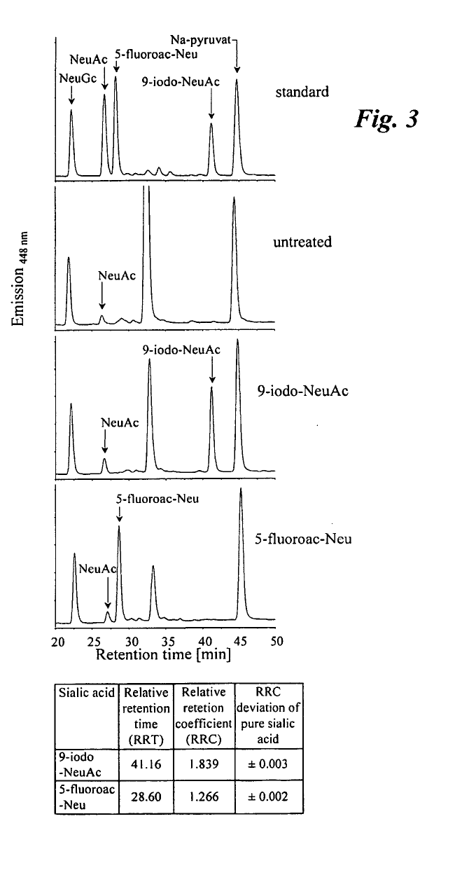

FIG. 3

Sialic acid analogues are incorporated in cell surface glycoconjugates. HPLC chromatograms of DMB-derivatised sialic acids hydrolysed from glycoproteins of sialic acid analogue-treated BJA-B K20 cells. (A) standards, (B) untreated cells, (C) cells cultivated in 5-N-fluoroacNeu, (D) cells cultivated in 9-iodo-NeuAc. Peaks were identified as follows: NeuGc (internal standard), NeuAc, 5-N-fluoroac-Neu, 9-iodo-NeuAc, Na-pyruvate (internal standard). Internal standards were added to each sample.

The unidentified peak at 34 min occured in all cell-derived samples.

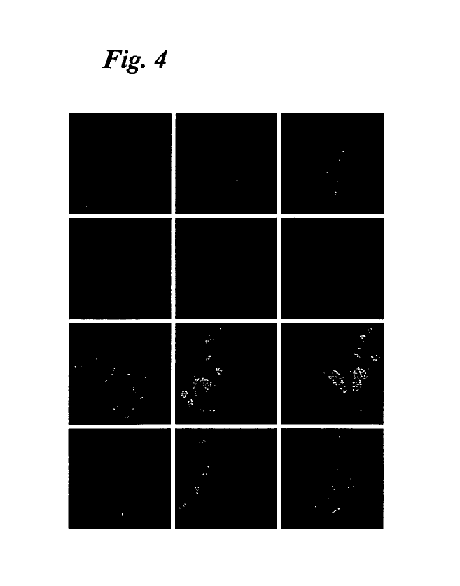

FIG. 4

Kinetics of 9-iodo-NeuAc-induced expression of sialoglycan CD75s in BJA-B K20 cells in comparison with its precursor 6-iodo-ManNAc. Cells were cultivated under serum-free conditions for at least 7 d and then either ManNAc, 6-iodo-ManNAc, 9-iodo-NeuAc or NeuAc (each 5 mM) was added. The de novo expression of CD75s was monitored at the indicated time points by indirect immunofluorescence using fixed and permeabilized cells. Representative confocal pictures of two independent experiments are shown.

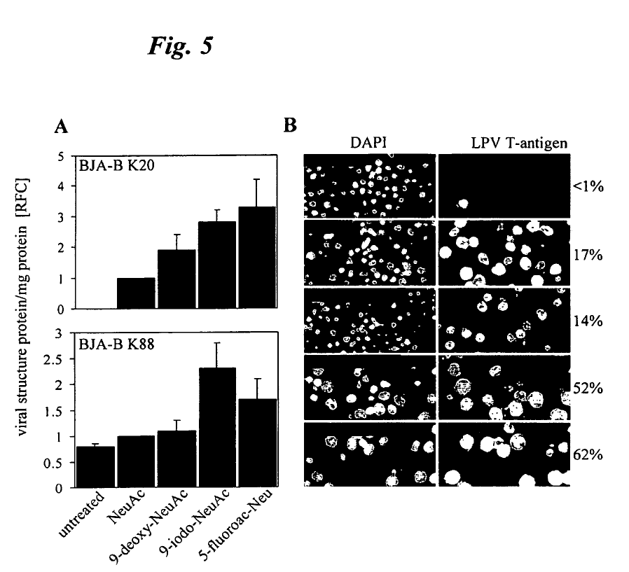

FIG. 5

Changes in permissivity for the sialic acid-dependent B-lymphotropic papovavirus (LPV) in hyposialylated (K20) and normally sialylated (K88) BJA-B cells after treatment with sialic acid analogues. Cells were treated with analogues or NeuAc for 3d. 50 h post LPV inoculation the LPV infection was quantified (A) by detecting the amount of LPV VP1 in cell lysates relative to the total protein content by ELISA and (B) by indirect immunofluorescence microscopy as percentage of LPV T-antigen-positive BJA-B K20 cells. A, values are given as arithmetic means±SD of three independent experiments. B, representatives of three experiments are shown. DAPI-staining (left) indicates the total amount of cells in the section, on the right LPV T-antigen positive cells are shown with the average percentage given beside.

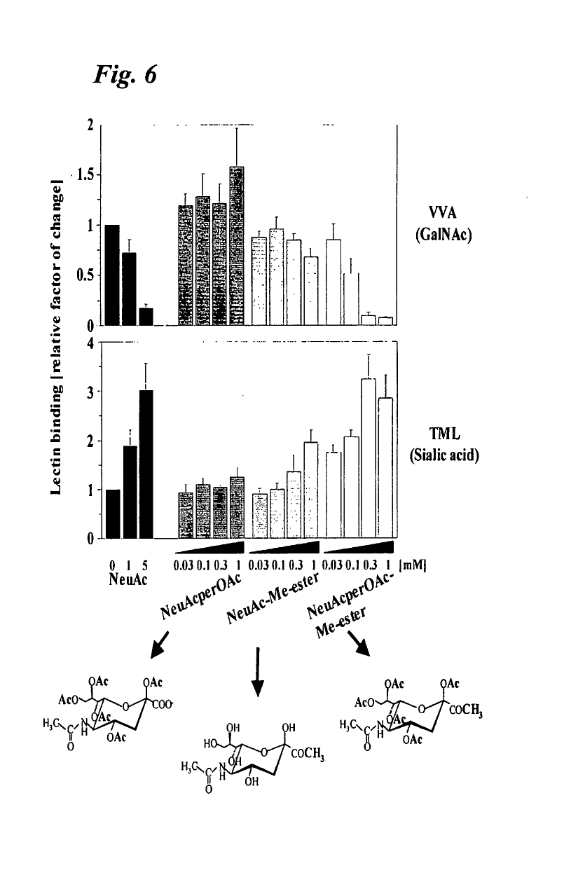

FIG. 6

Peracetylation of NeuAc only facilitates uptake if the carboxyl group is additionally esterified. Hyposialylated HL60-I cells were cultivated in the presence of indicated synthetic sialic acid analogue (stock solution 200 mM in DMSO) ranging from 0.03 to 1 mM or 0, 1 or 5 mM NeuA for 72 h with DMSO concentrations adjusted to 0.5%. Cells were stained with either directly fluorochrome-coupled lectin VVA, or biotin-coupled lectin (TML) and streptavidin-FITC for analysis by flow cytometry. Values given are relative factors of change of the mean fluorescence intensity of pretreated cells relative to untreated cells. The bars represent arithmetic means±SD (n=3). Molecular structures are given below the diagram.

Surprisingly it was found that synthetic sialic acid analogues can be incorporated into cell surface glycoconjugates. To test whether cells can incorporate synthetic sialic acid analogues from the culture medium, a metabolic complementation assay was performed on endogenously hyposialylated, UDP-GlcNAc-2-epimerase-deficient HL60-I cells.

Cells were cultivated for 24 h in the presence of one of 16 different synthetic sialic acid analogues, carrying distinct C5 or C9 modifications (FIG. 1), and subsequently, changes in cell surface sialylation were assessed by flow cytometry using three fluorochrome-conjugated sialic acid-sensitive lectins. Vicia villosa agglutinin (VVA) detects GalNAc residues, and its binding sites can be masked by the addition of terminal sialic acid residues. Limax flavus agglutinin (LFA) and Tritrichomonas mobilensis agglutinin (TLM) both detect terminal sialic acid residues regardless of their linkage, however, in vitro studies have revealed quite distinct binding affinities of LFA and TLM to different synthetic sialic acid analogues.

Medium supplementation with NeuAc, which served as a positive control, increased binding of LFA and TLM to HL60-I cells 9.5- and 4.8-fold, respectively (FIG. 2). Concomitantly, binding of VVA decreased markedly in NeuAc-treated cells compared to untreated controls, confirming an increased masking of penultimate GalNAc residues by sialic acid.

Of the 16 synthetic sialic acid analogues tested, 10 resulted in a significantly decreased VVA-binding to HL60-I cells (FIG. 2; analogues a, f, g, h, i, j, k, n, o, p). Most of these compounds also increased binding of LFA (except for k, n)) and of TLM (except for k).

The effect of synthetic analogues was similar to or less pronounced than that of NeuAc with the exception of 9-iodo-NeuAc, which most strongly increased binding of both LFA (19.2-fold) and TLM (6.4-fold). Two compounds, NeuAc-Me-Ester and, to a lesser extent NeuAc-Et-Ester, are unstable under physiological conditions and are most likely hydrolysed to NeuAc in the cytoplasm. This assumption is supported by the observation that their effects on LFA and TLM binding were very similar to those of NeuAc at equimolar concentrations (FIG. 2).

Of the six compounds, which had weak or no effect on VVA binding, four (analogues d, e, l, m) carried either a glycin- or a succinyl-residue, both very bulky substitutions, in position C5 or C9 (FIG. 2). 9-amino-NeuAc and 9-acetamino-NeuAc also failed to induce changes in cell surface sialylation.

To confirm that synthetic sialic acid analogues supplementing the medium were taken up and metabolically incorporated as analogues into surface sialoglyco-conjugates the membrane-associated sialic acid fraction was analyzed by High Performance Liquid Chromatography (HPLC).

UDP-GlcNAc 2-epimerase-deficient BJA-B K20 cells were first cultivated for at least 7 days under serum-free conditions to maximally deplete their physiological sialic acid pools (1,23) and then incubated with either 9-iodo-NeuAc or 5-fluoroac-Neu (each 5 mM) for 48 h.

Both synthetic sialic acid analogues could unambiguously be identified by HPLC in the glycoprotein-associated sialic acid fraction of pre-treated cells (FIG. 3). Both, 9-iodo-NeuAc (C) and 5-fluoroac-Neu (D) were eluted as discrete peaks that coincided with the peak of authentic 9-iodo-NeuAc and 5-fluoroac-Neu, respectively. An astounding 90.5% and 86.1% of membrane-associated sialic acids in K20 cells consisted of 5-fluoroac-Neu and 9-iodo-NeuAc, respectively. There was no increase in the amount of NeuAc as compared to cells cultured without sialic acid, indicating that no re-modification of these synthetic analogues resulting in NeuAc had occurred.

To exclude that carry-over of free sialic acid analogues could account for these results, one of the two substances were added for 48 h, while the other substance was added in an equimolar concentration for 1 min prior to cell harvest. Neither of the shortly added analogues (FIG. 3C, D) was detectable and thus the washing and purification procedure was validated.

The superiority of biosynthetic incorporation of sialic acid variants by NeuAc derivatives over derivatized ManNAc precurors was shown by directly comparing 9-iodo-NeuAc and the corresponding 6-iodo-ManNAc. K20 cells cultivated under serum-free conditions were incubated for 0-6 h with either 9-iodo-NeuAc or 6-iodo-ManNAc prior to analysis of CD75s expression. CD75s is an α-2,6 sialylated cell surface antigen of mature B-lymphocytes which is not detectable with the monoclonal antibody HH2 in K20 cells cultured in the absence of serum. 9-iodo-NeuAc treatment reconstituted the epitope, resulting in a strong intracellular signal already within 1 hour whereas 6-iodo-ManNAc did not show any effect within 6 h (FIG. 4). We conclude that presentation of C9-modified sialic acids on the cell surface is only possible by medium supplementation with sialic acid analogues, but not with ManNAc analogues.

It was further surprisingly found that specific synthetic sialic acid analogues restore LPV-permissivity even better than NeuAc. To study biological effects of the incorporated sialic acid analogues BJA-B cells were examined for permissivity to infection by the B-lymphotropic papovavirus (LPV), which uses a sialylated receptor. It was known that NeuAc treatment turns non-permissive BJA-B K20 cells into permissive cells, with about 17% of cells infected and viral capsid protein VP1 produced in amounts similar to that of similarly infected, permissive, untreated K88 cells. In K88 cells NeuAc treatment did not change permissivity significantly. Surprisingly, all three modified sialic acids tested increased LPV-permissivity of BJA-B K20 cells and K88 cells dramatically. With 9-iodo-NeuAc and 5-fluoroac-Neu VP1-expression was increased 2-to 3-fold as compared to NeuAc and more than 50% of the K20 cells were infected. Cells proliferated more slowly during infection and cytopathic effects of virus infection, such as enlarged nuclei were observed (FIG. 4B, DAPI-staining). Apparently, the increased VP1-expression is due to both increased number of infected cells and increased VP1-expression per cell. Although there could also be an influence of the sugars on intracellular virus replication, it is most likely that 9-iodo-NeuAc and 5-fluoroac-Neu and to a lesser extent 9-deoxy-NeuAc increased the efficacy of receptor-virus interaction resulting in multiple infections. However, the panel of sialic acids tested is too small to give a prediction on sialic acid residues important for LPV binding.

It was also surprisingly found that increased hydrophobicity facilitates sialic acid uptake. Sugars are very hydrophilic and it has been shown that increasing the hydrophobicity by peracetylation of hydroxyl groups enables or facilitates uptake of disaccharides (Collins, B. E., Fralich, T. J., Itonori, S., Ichikawa, Y. and Schnaar, R. L. (2000) Glycobiology, 10, 11-20; Sarkar, A. K., Fritz, T. A., Taylor, W. H. and Esko, J. D. (1995) Proc Natl Acad Sci USA, 92, 3323-7; Sarkar, A. K., Rostand, K. S., Jain, R. K., Matta, K. L. and Esko, J. D. (1997) J Biol Chem, 272, 25608-16). For ManNAc derivatives it has been shown that peracetylation reduces required medium concentrations (Collins et al., 2000). The acetyl groups are cleaved-off intracellularly by cellular esterases (Collins et al., 2000; Sarkar et al., 1995; Sarkar et al., 1997) and the derivatives are then processed as known for the non-acetylated sugars. In order to address the question whether peracetylation also facilitates uptake of N-acetylneuraminic acid, HL60-I cells were cultivated in the presence of peracetylated NeuAc at concentrations ranging from 0.03 mM to 1 mM for 3 days. Changes in cell surface sialylation were measured by flow cytometry using Vicia Villosa agglutinin (VVA) and Tritrichomonas mobilensis lectin (TML). As a positive control, cells cultivated in the presence of 1 or 5 mM NeuAc and showed significant changes in lectin binding at both concentrations. Surprisingly, treatment with completely peracetylated NeuAc (NeuAcperOAc) up to 1 mM had no effect on cell surface sialylation (FIG. 6). But esterification of the remaining free carboxyl group resulted in a substance (NeuAcperOAc-Me-ester) that strongly increased cell surface sialylation. Medium supplementation with 0.3 mM NeuAcperOAc-Me-ester showed similar effects as 5 mM NeuAc. This represents a reduction in concentration by more than 16-fold and is in a similar range as reported for peracetylated ManNAc and ManNAc (Collins et al., 2000). Esterification of the carboxyl group alone (NeuAc-Me-ester) did not affect uptake, both, NeuAc Me-ester and NeuAc treatment showed almost identical effects on sialylation at 1 mM (FIG. 6).

These surprising findings demonstrate the advantages of the present invention.

It is shown that synthetic sialic acid analogues can be readily taken up, metabolized and incorporated into cellular glycoconjugates of hyposialylated BJA-B K20 and HL60-I cells. As confirmed by HPLC for two analogues the incorporated, membrane-bound sialic acid was identical to the C5- or C9-substituted analogue added to the medium, and made up>85% of the sialic acid on the cell surface. When the effect of three analogues on the sialic acid-dependent infection of human BJA-B cells by the B-lymphotropic papovavirus (LPV) was studied a striking increase in LPV permissivity compared to NeuAc treatment was observed.

This novel system allows for a versatile and efficient biosynthetic modulation of surface sialylation in living cells, making possible detailed structure-function studies for a variety of sialic acid-dependent ligand-receptor interactions in their native context.

The glycoconjugates of the present invention can therefore be used in medical treatment of living beings, especially human beings. The treatment ranges from immunosuppression, cell protection against microbial infection, stimulation of hematopoesis, regulation of hormonal secretion and hormonal activation, but is not limited to these uses.

Substances and methods as given below were used according to the invention.

Cell Lines

Human B lymphoma cell line BJA-B (Burkitt's lymphoma-like, EBV-negative; (Menezes, J., Leibold, W., Klein, G., and Clements, G. (1975) Biomedicine 22(4), 276-84), BJA-B subclones K20 and K88 (Keppler, O. T., Hinderlich, S., Langner, J., Schwartz-Albiez, R., Reutter, W., and Pawlita, M. (1999) Science 284(5418), 1372-6; Keppler, O. T., Herrmann, M., Oppenländer, M., Meschede, W., and Pawlita, M. (1994) J Virol 68(11), 6933-9; Keppler, O. T., Peter, M. E., Hinderlich, S., Moldenhauer, G., Stehling, P., Schmitz, I., Schwartz-Albiez, R., Reutter, W., and Pawlita, M. (1999) Glycobiology 9(6), 557-69) and human myeloid leukemia cell line HL60 (34) variant HL60-I (Lowe, J. B., Stoolman, L. M., Nair, R. P., Larsen, R. D., Berhend, T. L., and Marks, R. M. (1990) Cell 63(3), 475-84) were propagated as suspension cultures in Erlenmeyer flasks with RPMI 1640 medium, supplemented with 10% heat-inactivated fetal calf serum (FCS) (GIBCO BRL, Eggenstein, Germany), 2 mM glutamine, 100 U/ml penicillin, and 100 U/ml streptomycin in a humidified 5% CO₂ atmosphere at 37° C.

For serum-free conditions cells were cultivated without FCS but with addition of Nutridoma-HU at the concentration suggested by the manufacturer (Roche Diagnostics, Mannheim, Germany) for at least 7 days prior to an experiment.

In sugar supplementation assays, cells seeded at 5×10⁵ cells/ml in culture medium buffered with 40 mM HEPES (pH 7.2) were cultivated for 24 h in the presence or absence of 5 mM sialic acids (stocks of 100 mM dissolved in H₂O, stored at 4° C.).

For HPLC analysis serum starved HL60-I cells were incubated with 5 mM 9-iodo NeuAc for 48 h in the presence of 40 mM HEPES pH 7.2 while 5-fluoroac Neu was added directly before harvesting cells as a negative control or vice versa.

Flow Cytometry

Lyophilized FITC-conjugated lectins Vicia villosa (VVA) was obtained from Sigma, dissolved in H₂O (1 mg/ml), aliquoted and stored at −20° C. according to the manufacturer's instructions. FITC-conjugated Limax flavus agglutinin (LFA) was from EY Laboratories (San Manteo, Calif., USA) and biotin-coupled Tritrichomonas mobilensis agglutinin (TLM) from Calbiochem. Lectin staining procedure and fluorescence-activated cell scanning on a Becton Dickinson FACScan cytometer using Cellquest II software were carried out as described (Keppler, O. T., Molden-hauer, G., Oppenländer, M., Schwartz-Albiez, R., Berger, E. G., Funderud, S., and Pawlita, M. (1992) Eur J Immunol 22(11), 2777-81). Briefly, cells (3×10⁵) were washed twice in cold PBS and then incubated in 100 μl PBS/0.05% NaN3, containing either fluorochrome-conjugated lectins VVA (50 μg/ml), LFA (20 μg/ml), or biotinylated TLM (20 μg/ml) for 45 min on ice in the dark. For biotinylated TLM a secondary incubation step with steptavidin-FITC (20 μg/ml) (Sigma) for 30 min on ice in the dark is required. After washing with PBS, cells were resuspended in 300 μl PBS and analyzed by flow cytometry.

Detection of Incorporated Sialic Acids Via HPLC

To confirm that the modifications are still present after incorporation into glycoconjugates sialic acids were cleaved off cell surfaces and analyzed by HPLC. Cells (1×10⁷) were washed twice with PBS, frozen at −20° C. and lysed by hypotonic shock in distilled water in an ultrasonic bath (5 min, 4° C.). The crude membrane fraction was pelleted by centrifugation at 10,000×g for 15 min and the pellet was washed twice with distilled water. The lyophilized pellet was delipidated by stepwise washing three times with 600 μl chloroform/methanol (2:1, 1:1, 1:2 by volume) for 5 min in an ultrasonic bath. After centrifugation at 10,000×g (15 min, 4° C.) the dry pellet was hydrolyzed for 1 h with 200 μl of 2 M acetic acid at 80° C. (Hara, S., Takemori, Y., Yamaguchi, M., Nakamura, M., and Ohkura, Y. (1987) Anal Biochem 164(1), 138-45) the acetic acid was evaporated after separation. After washing and resuspending in H₂O, sample was further purified using a cation-exchange column (AG-50W-X12, H+-form, 100-200 mesh) (BiORad, Muenchen, Germany), lyophilized and resuspendet in 100 μl H₂O. Sialic acids derivation was performed according to a method as given below and samples were analyzed on a reversed-phase C18 column as described (Keppler, O. T., Stehling, P., Herrmann, M., Kayser, H., Grunow, D., Reutter, W., and Pawlita, M. (1995) J Biol Chem 270(3), 1308-14).

Immunofluorescence Microscopy

Cells adherent to poly-L-lysin (Sigma) coated slides were fixed with 3% (w/v) paraformaldehyde, 2% (w/v) sacchrose in PBS for 10 min and blocked with 10 mg/ml glycine in PBS for 10 min. After incubation with monoclonal CD75s antibody HH2 (5 μg/ml) in IF-buffer (0.5 mM MgCl2, 1 mM CaCl₂, 0.2% (w/v) gelatine, 0.1% saponine (w/v) (ICN, Eschwege, Germany) in PBS) for 60 min, cells were washed twice with PBS and then incubated with goat anti-mouse IgM-Cy3 (0.2 μg/ml) Dianova, Hamburg, Germany) in IF-buffer for 60 min. Washed slides were mounted with elvanol. Digital pictures were aquired using a confocal laser scanning microscope (Leica DM IRBE) and TCS NT software.

LPV Infection

BJA-B subclones were infected with the B-lymphotropic papovavirus (LPV) as described (Keppler, O. T., Herrmann, M., Oppenländer, M., Meschede, W., and Pawlita, M. (1994) J Virol 68(11), 6933-9). Addition of HEPES inhibited LPV infection and was therefore omitted without significantly affecting cell viability. LPV infection was quantified by indirect immunofluorescence microscopy as percentage of LPV T-antigen positive cells and the amount of LPV VP1 in cell lysates was determined by ELISA relative to the total protein extracted.

Synthesis of Sialic Acid Derivates and Analogues:

Sialic acid derivates and analogues according to the present invention are compiled in FIG. 1. These compounds have been synthesized in a usual manner using standard synthetic pathways.

9-deoxy derivative (a) (5-N-acetyl-9-deoxy-neuraminic acid) was obtained by catalytic hydrogenation of the corresponding 9-iodo compound (Brossmer, R., and Gross, H. J. (1994) Methods Enzymol 247, 153-76). Synthesis of 9-amino-NeuAc (b) (5-N-acetyl-9-amino-9-deoxy-neuraminic acid) has been described (Isecke, R. (1994)). 9-acetamido-NeuAc (c) (5-N-acetyl-9-acetamido-9-deoxy-neuraminic acid) and 9-N-Succ-NeuAc (e) (5-N-acetyl-9-deoxy-9-succinylamido-neuraminic acid) were produced from (b) by acetylation (Brossmer, R., and Gross, H. J. (1994) Methods Enzymol 247, 153-76) and succinylation, respectively. Reaction of (b) with N-benzyloxycarbonyl glycine 4-nitrophenyl ester followed by catalytic hydrogenation afforded 9-N-Gly-NeuAc (d) (9-N-aminoacetamido-9-deoxy-neuraminic acid). 9-iodo-NeuAc (f) (5-N-acetyl-9-deoxy-9-iodo-neuraminic acid) was prepared by conversion of the 9-O-tosylate with sodium iodide in dimethylformamide or, much simpler, employing Mitsunobu conditions. Synthesis of 9-thio-NeuAc (g) (5-N-acetyl-9-deoxy-9-thio-neuraminic acid) which is the starting compound of other sulphur containing analogues has been reported (Itoh, M., Hetterich, P., Isecke, R., Brossmer, R., and Klenk, H. D. (1995) Virology 212(2), 340-7). Reaction of (g) with methyl iodide gave 9-SCH3-NeuAc (h) (5-N-acetyl-9-deoxy-9-methylthio-neuraminic acid). Oxidation of (h) using peroxybenzoic acid afforded 9-SO2CH3-NeuAc (i) (5-N-acetyl-9-deoxy-9-methylsulfonyl-neuraminic acid).

For the synthesis of 5-fluorac-Neu (j) (5-N-fluoroacetyl-neuraminic acid) and 5-trifluoroac-Neu (k) (5-N-trifluoroacetyl-neuraminic acid), methyl α- or benzyl α-glycoside of neuraminic acid (free NH₂-group at C-5) was reacted with the respective acid anhydride. Subsequent controlled acid hydrolysis of the methylglycoside or catalytic hydrogenation of the benzylglycoside afforded the desired compounds, 5-N-thioac-Neu (n) (5-N-thioacetyl-neuraminic acid) was prepared as described (Isecke, R. and Brossmer, R. (1994) Tetrahedron 50, 7445-7460). Reaction of benzyl α-glycoside of neuraminic acid with the 4-nitrophenyl ester of N-benzyloxycarbonyl glycerine followed by catalytic hydrogenation produced 5-N-Gly-Neu (l) (5-N-aminoacetyl-neuraminic acid) (Brossmer, R., and Gross, H. J. (1994) Methods Enzymol 247, 153-76). Synthesis of 5-N-Succ-NeuAc (m) (5-N-succinyl-neuraminic acid) was performed by acid hydrolysis of the corresponding methyl glycoside, NeuAc-Me-ester (O) (methyl N-acetyl neuraminate) and NeuAc-Et-ester (p) (ethyl N-acetyl neuraminate) were obtained by acid catalysed (preferably cation exchange resin) esterification.

Acetylation of sugars was performed in a known manner according to standard procedures with acetic anhydride in pyridine for 1 h at ambient temperature (Sarkar, A. K., Fritz, T. A., Taylor, W. H., and Esko, J. D. (1995) Proc. Natl. Acad. Sci. U.S.A. 92, 3323-3327; Collins, B. E., Fralich, T. J., Itonori, S., Ichikawa, Y., and Schnaar, R. L. (2000) Glycobiology 10, 11-20). Compounds were purified by silica gel column chromatography using stepwise elution with toluene, toluene-ethanol (50:1), and toluene-ethanol (20:1) as eluants.

All synthesized sialic acid analogues and derivates were characterized by high resolution nuclear magnetic resonance spectroscopy and fast atom bombardment mass spectrometry. The proposed structures were confirmed by the analyses.

1. Glycoconjugates obtained by conjugating a sialic acid derivate of general formula I to a mono-, di- or oligosaccharide with up to 40 glycosidically linked, optionally branched sugar residues representing furanose and/or pyranose rings, which are linked N- or O-glycosidically to a polypeptide 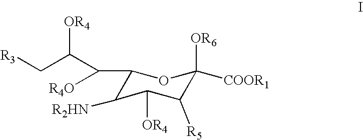
wherein
R1 represents hydrogen or lower alkyl up to 5 carbon atoms, which may be branched, unbranched, acyclic, alicyclic or cyclic,
R2 is acetyl, thioacetyl or succinyl, which can be substituted with up to 3 fluoro atoms or an amino group, or an acyl or thioacyl group with up to 5 carbon atoms in the alkyl moiety, which may be branched, unbranched, acyclic, alicyclic or cyclic,
R3 is a halogen atom, a methylsulfide group, a methylsulfate group or acyl or thioacyl group with up to 5 carbon atoms in the alkyl moiety, which may be branched, unbranched, acyclic, alicyclic or cyclic,
R4 represents, independently from each other, hydrogen, acetyl, thioacetyl or succinyl, which can be substituted with up to 3 fluoro atoms or an amino group, or an acyl or thioacyl group with up to 5 carbon atoms in the alkyl moiety, which may be branched, unbranched, acyclic, alicyclic or cyclic,
R5 is hydrogen or a halogen atom
R6 represents hydrogen, acetyl, thioacetyl or succinyl, which can be substituted with up to 3 fluoro atoms or an amino group, or an acyl or thioacyl with up to 5 carbon atoms in the alkyl moiety, which may be branched, unbranched, acyclic, alicyclic or cyclic and wherein the sialic acid derivate of general formula I is conjugated to said mono-, di- or oligosaccharide via R6.

2. Glycoconjugates according to claim 1,
wherein
R1 represents hydrogen or lower alkyl up to 3 carbon atoms, which may be branched, unbranched or cyclic,
R2 is acetyl,
R3 represents, independently from each other, hydrogen or acetyl,
R4 represents, independently from each other, hydrogen or acetyl and
R5 is hydrogen.

3. Glycoconjugates according to claim 1,
wherein
R1 represents hydrogen, methyl or ethyl,
R2 is acetyl,
R3 represents, independently from each other, hydrogen or acetyl,
R4 represents, independently from each other, hydrogen or acetyl and
R5 is hydrogen.

4. Glycoconjugates according to claim 1,
wherein
the sialic acid derivate to be incorporated is selected from
5-N-acetyl-9-deoxy-neuraminic acid,
5-N-acetyl-9-deoxy-neuraminic acid peracetylate,
5-N-acetyl-9-deoxy-neuraminic acid methyl ester peracetylate,
5-N-acetyl-9-deoxy-neuraminic acid ethyl ester peracetylate,
5-N-acetyl-9-amino-9-deoxy-neuraminic acid,
5-N-acetyl-9-amino-9-deoxy-neuraminic acid peracetylate,
5-N-acetyl-9-amino-9-deoxy-neuraminic acid methyl ester peracetylate,
5-N-acetyl-9-amino-9-deoxy-neuraminic acid ethyl ester peracetylate,
5-N-acetyl-9-acetamido-9-deoxy-neuraminic acid,
5-N-acetyl-9-acetamido-9-deoxy-neuraminic acid peracetylate,
5-N-acetyl-9-acetamido-9-deoxy-neuraminic acid methyl ester peracetylate,
5-N-acetyl-9-acetamido-9-deoxy-neuraminic acid ethyl ester peracetylate,
5-N-acetyl-9-aminoacetamido-9-deoxy-neuraminic acid,
5-N-acetyl-9-aminoacetamido-9-deoxy-neuraminic acid peracetylate,
5-N-acetyl-9-aminoacetamido-9-deoxy-neuraminic acid methyl ester peracetylate,
5-N-acetyl-9-aminoacetamido-9-deoxy-neuraminic acid ethyl ester peracetylate,
5-N-acetyl-9-deoxy-9-succinylamido-neuraminic acid,
5-N-acetyl-9-deoxy-9-succinylamido-neuraminic acid peracetylate,
5-N-acetyl-9-deoxy-9-succinylamido-neuraminic acid methyl ester peracetylate,
5-N-acetyl-9-deoxy-9-succinylamido-neuraminic acid ethyl ester peracetylate,
5-N-acetyl-9-deoxy-9-iodo-neuraminic acid,
5-N-acetyl-9-deoxy-9-iodo-neuraminic acid peracetylate,
5-N-acetyl-9-deoxy-9-iodo-neuraminic acid methyl ester peracetylate,
5-N-acetyl-9-deoxy-9-iodo-neuraminic acid ethyl ester peracetylate,
5-N-acetyl-9-deoxy-9-thio-neuraminic acid,
5-N-acetyl-9-deoxy-9-thio-neuraminic acid ester peracetylate,
5-N-acetyl-9-deoxy-9-thio-neuraminic acid methyl ester peracetylate,
5-N-acetyl-9-deoxy-9-thio-neuraminic acid ethyl ester peracetylate,
5-N-acetyl-9-deoxy-9-methylthio-neuraminic acid,
5-N-acetyl-9-deoxy-9-methylthio-neuraminic acid peracetylate,
5-N-acetyl-9-deoxy-9-methylthio-neuraminic acid methyl ester peracetylate,
5-N-acetyl-9-deoxy-9-methylthio-neuraminic acid ethyl ester peracetylate,
5-N-acetyl-9-deoxy-9-methylsulfonyl-neuraminic acid,
5-N-acetyl-9-deoxy-9-methylsulfonyl-neuraminic acid peracetylate,
5-N-acetyl-9-deoxy-9-methylsulfonyl-neuraminic acid methyl ester peracetylate,
5-N-acetyl-9-deoxy-9-methylsulfonyl-neuraminic acid ethyl ester peracetylate,
5-N-flouroacetyl-neuraminic acid peracetylate,
5-N-flouroacetyl-neuraminic acid methyl ester peracetylate,
5-N-flouroacetyl-neuraminic acid ethyl ester peracetylate,
5-N-triflouroacetyl-neuraminic acid peracetylate,
5-N-triflouroacetyl-neuraminic acid methyl ester peracetylate,
5-N-triflouroacetyl-neuraminic acid ethyl ester peracetylate,
5-N-aminoacetyl-neuraminic acid peracetylate,
5-N-aminoacetyl-neuraminic acid methyl ester peracetylate,
5-N-aminoacetyl-neuraminic acid ethyl ester peracetylate,
5-N-succinyl-neuraminic acid peracetylate,
5-N-succinyl-neuraminic acid methyl ester peracetylate,
5-N-succinyl-neuraminic acid ethyl ester peracetylate,
5-N-thioacetyl-neuraminic acid peracetylate,
5-N-thioacetyl-neuraminic acid methyl ester peracetylate,
5-N-thioacetyl-neuraminic acid ethyl ester peracetylate,
5-N-acetyl-9-deoxy-neuraminic acid methyl ester,
5-N-acetyl-9-deoxy-neuraminic acid ethyl ester,
5-N-acetyl-9-deoxy-neuraminic acid methyl ester peracetylate,
5-N-acetyl-9-deoxy-neuraminic acid ethyl ester peracetylate,
5-N-acetyl-9-deoxy-neuraminic acid peracetylate,
5-N-acetyl-9-deoxy-9-iodo-neuraminic acid methyl ester,
5-N-acetyl-9-deoxy-9-iodo-neuraminic acid, methyl ester peracetylate.

5. Method for the production of glycoconjugates according to claim 1, characterized in that a sialic derivative of general formula I as given in claim 2, 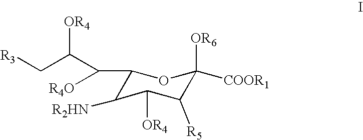
wherein
R1 represents hydrogen or lower alkyl up to 5 carbon atoms, which may be branched, unbranched, acyclic, alicyclic or cyclic,
R2 is acetyl, thioacetyl or succinyl, which can be substituted with up to 3 fluoro atoms or an amino group, or an acyl or thioacyl group with up to 5 carbon atoms in the alkyl moiety, which may be branched, unbranched, acyclic, alicyclic or cyclic,
R3 is a halogen atom, a methylsulfide group, a methylsulfate group or acyl or thioacyl group with up to 5 carbon atoms in the alkyl moiety, which may be branched, unbranched, acyclic, alicyclic or cyclic.
R4 represents, independently from each other, hydrogen, acetyl, thioacetyl or succinyl, which can be substituted with up to 3 fluoro atoms or an amino group, or an acyl or thioacyl group with up to 5 carbon atoms in the alkyl moiety, which may be branched, unbranched, acyclic, alicyclic or cyclic,
R5 is hydrogen or a halogen atom and
R6 represents hydrogen, acetyl, thioacetyl or succinyl, which can be substituted with up to 3 fluoro atoms or an amino group, or an acyl or thioacyl group with up to 5 carbon atoms in the alkyl moiety, which may be branched unbranched, acyclic, alicyclic or cyclic,
is incorporated into a living body, especially to a mammal or cells derived from mammals and lower eukaryotes.

6. (Canceled).

7. Use of a sialic derivative of general formula I as given in any of the claims 1 to 5, for producing a pharmaceutical composition, for immunosuppression, cell protection, stimulation of hematopoesis regulation of hormonal secretion and hormonal activation.
